(19) United States
(12) Patent Application Publication
(10) Pub. No.: US 2007/0254333 A1
(43) Pub. Date: Nov. 1, 2007

(54) Novel pyranose stereoisomers and method for producing unnatural sugars via palladium catalyzed glycosylation

(76) Inventor: George Augustine O'Doherty, Morgantown, WV (US)

Correspondence Address: WEST VIRGINIA UNIVERSITY RESEARCH CORPORATION, 886 CHESTNUT RIDGE ROAD, P.O. BOX 6216, MORGANTOWN, WV, 26506-6216, US

(21) Appl. No.: 11/796,087

(22) Filed: Apr. 26, 2007

Related U.S. Application Data
(60) Provisional application No. 60/794,982, filed on Apr. 26, 2006.

(51) Int. Cl. C12P 33/00; C12P 19/04

(52) U.S. Cl. 435/52; 435/101

(57) ABSTRACT

The present invention can be a method to prepare a glycosylated natural product or pharmaceutical from either a BocO-pyranone or a BocO-enone. The identical methods of production utilize catalytic palladium and catalytic amounts of phosphine ligand to place the desired group stereospecifically at the C-1 position. The natural product can be the compound digitoxin. Post-glycosylation reactions can add H, OH, F, Cl, Br, I, NH₂, NHAc, CN, or N₃ on C-2, C-3, or C-4 and C-6 can be any alkyl, aryl, heteroalkyl, hydroxyalkyl groups with any amino, halo or hydroxyl substitution. A further aspect of the present invention can be a method to prepare either BocO-pyranone or BocO-enone. Another aspect of the present invention can be the 32 possible pyranose stereoisomers at C-1, C-2, C-3, C-4, or C-6 with substitutes chosen from H, OH, F, Cl, Br, I, NH₂, NHAc, CN, or N₃ on C-2, C-3, or C-4 and C-6 can be any alkyl, aryl, heteroalkyl, hydroxyalkyl groups with any amino, halo or hydroxyl substitution attached to an unnatural sugar attached to a natural product. Further the natural product can be digitoxigenin.

### CROSS-REFERENCE TO RELATED APPLICATIONS

This application claims priority from application 60/794982

### REFERENCE TO SEQUENCE LISTING, A TABLE, OR A COMPUTER PROGRAM LISTING COMPACT DISC APPENDIX

Not Applicable

### BACKGROUND OF THE INVENTION

Compounds bearing rare sugars have played a pivotal role in many pharmalogically important antibiotics, vaccines and antitumor agents. For instance, the cardiac glycoside digitoxin, which possesses both potent cardiac and anticancer activity, is the combination of two biologically inactive natural products, the aglycon digitoxigenin and the trisaccharide digoxose.

In contrast to typical glycosylation reactions, which install a fully functionalized sugar without consistent stereocontrol, this palladium catalyzed glycosylation reaction stereospecifically installs a pyranone/enone sugar/C-glycoside precursor. These pyranone/enone products can, via various post-glycosylation reactions, be converted into a myriad of substituted sugars. This approach allows for the synthesis of rare and unnatural sugars, which are not available by traditional glycosylation methods.

### BRIEF SUMMARY OF THE INVENTION

The following are definitions for terms within this application: NPOH is a hydroxyl group containing natural product and/or a pharmaceutical such as an alcohol or phenol.

A natural product can be any compound that is produced by a living organism's secondary metabolism. An NPO is a hydroxyl group containing natural product or pharmaceutical minus the hydrogen atom from the NPOH such as an alcohol, aromatic alcohol or heteroaromatic alcohol.

An aglycon is a noncarbohydrate group, such as an alcohol or phenol, combined with a sugar to form a glycoside and in this application includes the NPOH.

An aspect of the present invention is a method to prepare biologically active glycosylated compounds such as glycosylated natural products and an NPO attached to a sugar from BocO-pyranones using palladium/phosphine catalysis where the aglycon stereospecifically replaces the BocO group at the C-1 position of a corresponding pyranone. In addition, an R group can be placed at the C-6 position of the pyranose where the R group can be any alkyl, aryl, heteroalkyl, or hydroxyalkyl groups with any amino, halo or hydroxyl substitution. Further, the starting aglycon group can be natural products like digitoxigenin or the aglycon of digitoxin, which when glycosylated become significantly more potent anticancer compounds.

A further aspect of the present invention are pyranone post-glycosylation reactions to add X, Y, and Z to the C-2, C-3, and C-4s respectively. X, Y, or Z can be an H, OH, F, Cl, Br, I, NH₂, NHAc, CN, N₃, or OR, where R can be any alkyl, aryl, heteroalkyl, hydroxyalkyl groups with any amino, halo or hydroxyl substitution.

Another aspect of the present invention can be the 32 possible pyranose stereoisomers at C-1, C-2, C-3, C-4, or C-6 with substitutes chosen from H, OH, F, Cl, Br, I, NH₂, NHAc, CN, N₃, or OR, where R can be any alkyl, aryl, heteroalkyl, hydroxyalkyl groups with any amino, halo or hydroxyl substitution. Further, the starting aglycon group can be natural products like digitoxigenin, the aglycon of digitoxin, which when glycosylated become significantly more potent anticancer compounds.

An aspect of the present invention is a method to prepare glycosylated natural products (NPOH) from BocO-enone using palladium/phosphine catalysis where an NPO group is stereospecifically placed at the C-1 position of a corresponding pyranone. In addition an R group can be placed at the C-6 position where the R group can be any alkyl, aryl, heteroalkyl, hydroxyalkyl groups with any amino, halo or hydroxyl substitution. Further, the starting aglycon group can be natural products like digitoxigenin, the aglycon of digitoxin, which when glycosylated become significantly more potent anticancer compounds.

A further aspect of the present invention are pyranone post-glycosylation reactions which add X, Y, and Z to the C-2, C-3, and C-4s respectively and can be an H, OH, F, Cl, Br, I, NH₂, NHAc, CN, or N₃.

In addition the present invention comprises a method to allow any user to produce the C-1 BocO-pyranone with any desired C-6 R group from the group of alkyl, aryl, heteroalkyl, hydroxyalkyl groups with any amino, halo or hydroxyl substitution from a furan alcohol which in turn can be prepared from furan and a carboxylic acid.

A further aspect of the present invention is the use of the same palladium catalyzed glycosylation and post-glycosylation reactions for the installation of analogous cyclitol sugars with X, Y, and Z at the C-2, C-3, and C-4s respectively which can be an H, OH, F, Cl, Br, I, NH₂, NHAc, CN, or N₃. and a C-6 R group from the group of alkyl, aryl, heteroalkyl, hydroxyalkyl groups with any amino, halo or hydroxyl substitution.

The present invention also provides a method to prepare a C-1 BocO-enone from quinic acid and allows a user to prepare a L-cyclitol donor with a C-6 R group from the group of alkyl, aryl, heteroalkyl, hydroxyalkyl groups with any amino, halo or hydroxyl substitution.

### BRIEF DESCRIPTION OF THE SEVERAL VEIWS OF THE DRAWINGS

### DETAILED DESCRIPTION OF THE INVENTION

The invention can be a method for the preparation of glycosylated natural products or pharmaceuticals via a palladium catalyzed glycosylation of a hydroxyl group in the NPOH and a corresponding C-1 BocO-pyranone. The reaction stereospecifically converts the C-1 BocO group to an NPO group. The NPOH is treated with 1-2 equivalents of a given BocO-pyranone and catalytic amounts of palladium(0) and phosphine ligand in a 1:2 ratio. These reactions gives good yields (60% to 90%) of the corresponding pyranones with an NPO group stereospecifically installed at the C-1 position. The catalytic amount of the palladium(0)/ligand complex used ranges from 0.01% to about 20% while the ratio of palladium to phosphine ligand of the complex remains 1:2.

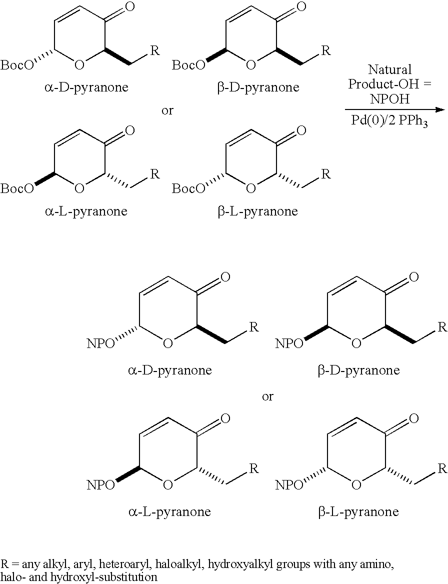

Various post-glycosylation reactions can in 1-5 steps convert the enone functionality of the pyranones to their corresponding glycosylated digitoxin analogous with the various pyranose sugars. These include various addition, oxidation, reduction and/or substitution reactions on the enone functionality. For example the C-4 ketone can be selectively reduced (e.g., with NaBH₄, 90%) to an allylic alcohol, which in turn can be dihydroxylated (e.g., OsO₄, 90%) to a manno-triol (X, Y, Z=OH). Alternatively, the allylic alcohol products can be reductively rearranged (e.g., NBSH/DIAD/PPh₃, 60-80%) before the dihydroxylation (e.g., OsO₄, 90%) to give a C-2 deoxy-allo-sugar (X═H, Y, Z=OH). By introducing various substitution reactions the hydroxyl groups can be converted into halides and/or azides, which can then reduced to amines and amides. One skilled in the art would be able to add X, Y, and Z to the C-2, C-3, and C-4s respectively. X, Y, or Z can be an H, OH, F, Cl, Br, I, NH₂, NHAc, CN, N₃, or OR, where R can be any alkyl, aryl, heteroalkyl, hydroxyalkyl groups with any amino, halo or hydroxyl substitution.

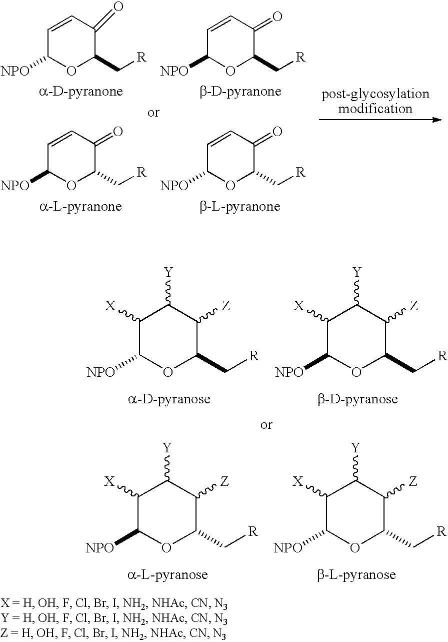

The method for the preparation of a glycosylated NPOH can be utilized to prepare unnatural cardiac glycosides such as digitoxin sugar analogues. Palladium catalyzed glycosylation of digitoxigenin, the digitoxin aglycon, and the corresponding C-1 BocO-pyranone stereospecifically converts the C-1 BocO group to a DigO group. The method is the same as before. Digitoxigenin is treated with 1-2 equivalents of a given BocO-pyranone and catalytic amounts of a palladium(0) and phosphine ligand in a 1:2 ratio. The catalytic amount of palladium (0) ranges from 0.01% to about 20% while the amount of ligand used range from about 0.02% to about 40%. The reaction gives good yields of around 60% to 90% of the corresponding pyranones with a DigO group stereospecifically installed at the C-1 position. As before various post-glycosylation reactions such as addition, oxidation, reduction and/or substitution reactions can convert the enone functionality of the pyranones to their corresponding glycosylated digitoxin analogue with the various pyranose sugars. One skilled in the art would be able to add X, Y, and Z to the C-2, C-3, and C-4s respectively. X, Y, or Z can be an H, OH, F, Cl, Br, I, NH₂, NHAc, CN, N₃, or OR, where R can be any alkyl, aryl, heteroalkyl, hydroxyalkyl groups with any amino, halo or hydroxyl substitution.

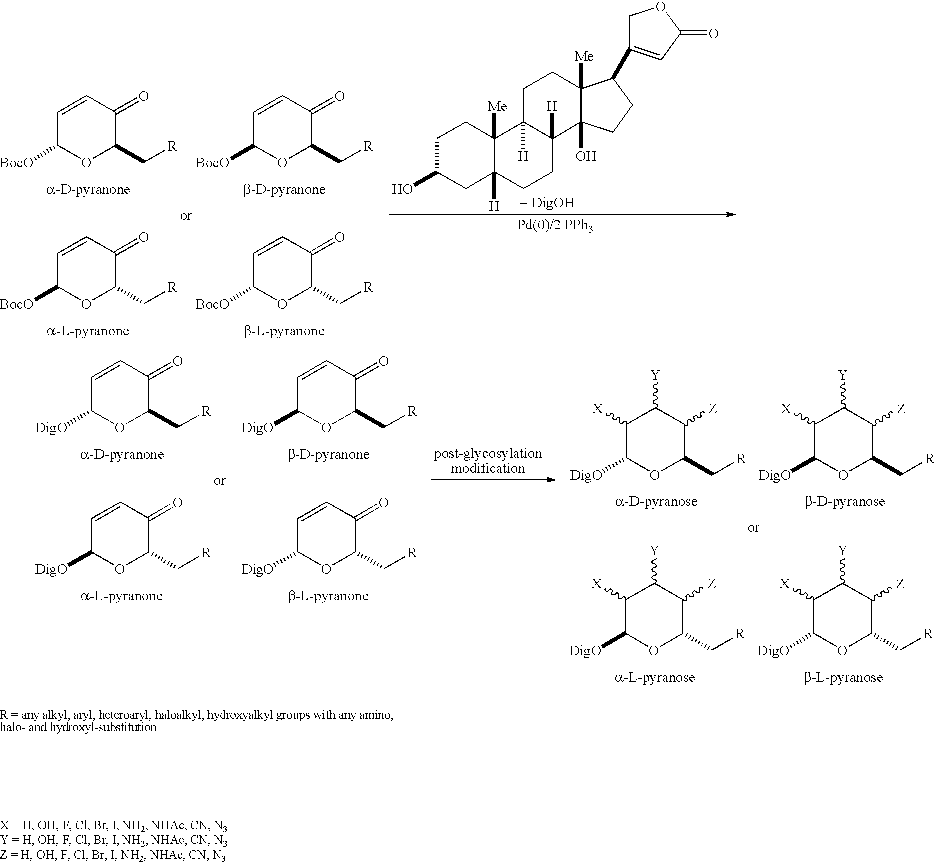

The method of the invention can yield 32 possible pyranose stereoisomers, including both (R) and (S) at C-1, C-2, C-3, C-4, and C-5, wherein the C-2 (X), C-3 (Y) and C-4 (Z) groups being any combination of the following substituents: H, OH, F, Cl, Br, I, NH₂, NHAc, CN, N₃, and the C-6 substituent being any alkyl, aryl, heteroaryl and alkyl group with any amino, halo- and hydroxyl-substitution.

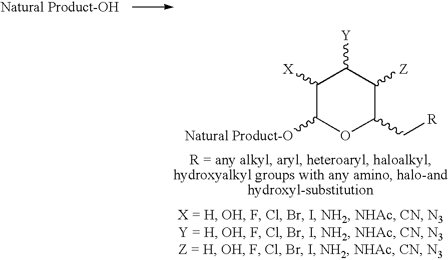

The natural product of the NPOH can be digitoxigenin, the aglycon of the cardiac glycoside digitoxin. The result is the aglycon being attached to 32 possible pyranose stereoisomers, both (R) and (S), at C-1, C-2, C-3, C-4, and C-5 wherein the C-2 (X), C-3 (Y) and C-4 (Z) groups being any combination of the substituents H, OH, F, Cl, Br, I, NH₂, NHAc, CN, N₃, and the C-6 substituent being any alkyl, aryl, heteroaryl and alkyl group with any amino, halo- and hydroxyl-substitution

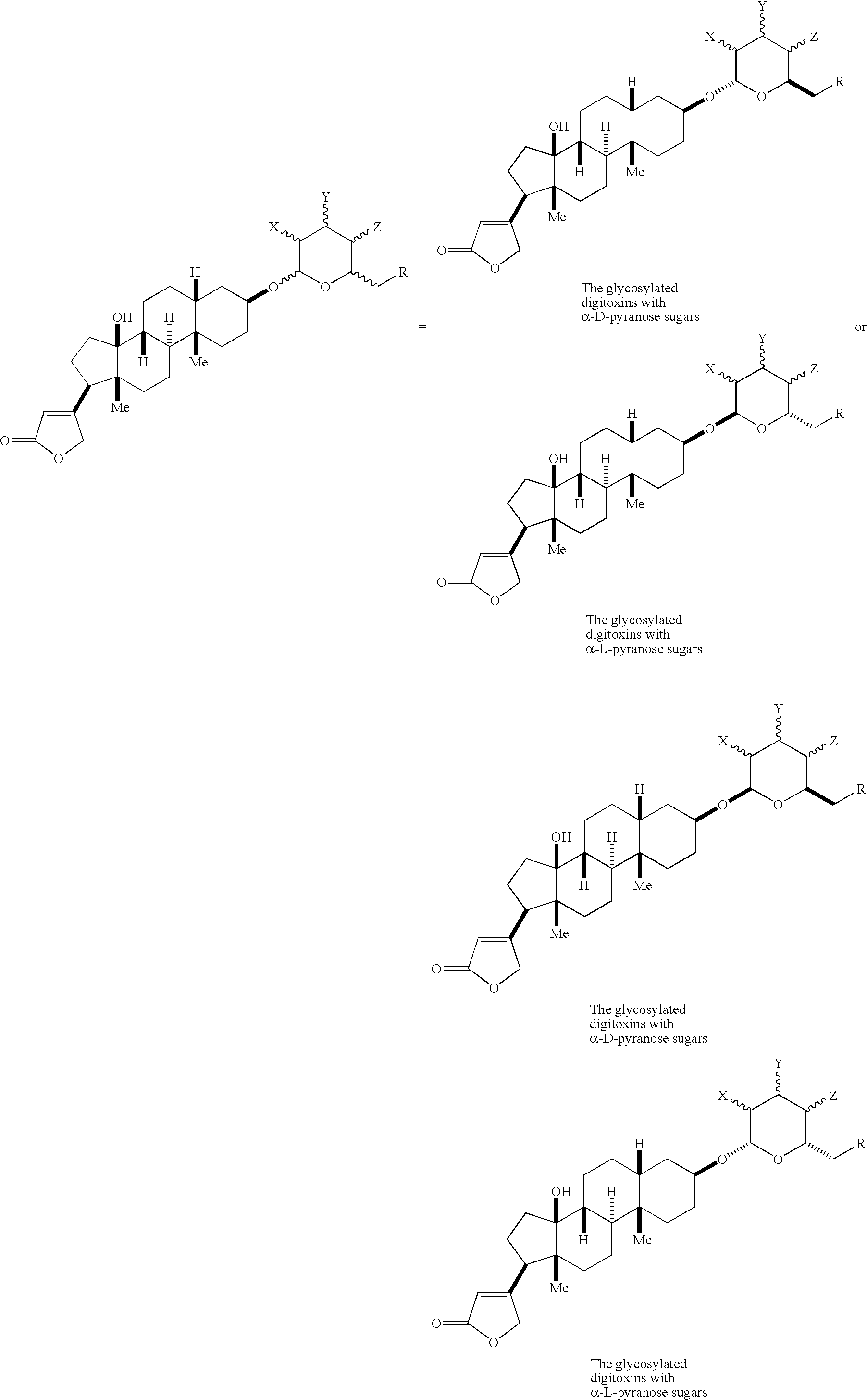

In addition, the same method can also be used for the preparation of the C-glycosylated NPOH via a palladium catalyzed glycosylation of an aglycon OH group and the corresponding C-1 BocO-enone. In this case, the C-1 BocO group is stereospecifically converted to an NPO group. Thus, natural products and/or pharmaceuticals can be treated with 1-2 equivalents of a given BocO-enone and catalytic amounts of palladium(0) and triphenylphosphine in a 1:2 ratio. The result are good yields (60% to 70%) of the corresponding enones with an NPO group at the C-1 position are stereospecifically produced. The catalytic amount of palladium(0) ranges from about 0.01% to about 20% while the amount of phosphine ranges from about 0.02% to about 40%.

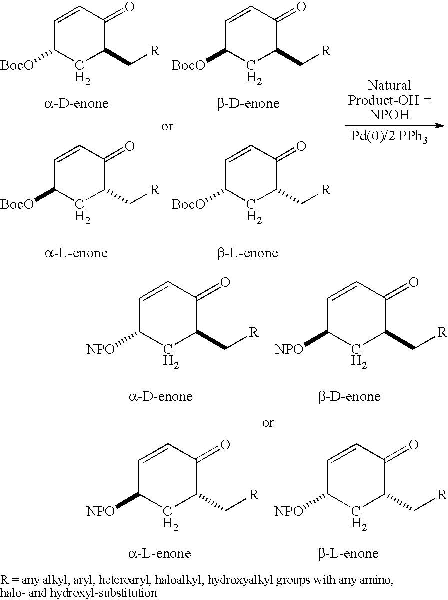

The same above mentioned post-glycosylation addition, oxidation, reduction and/or substitution reactions can in 1-5 steps convert the enone products to their corresponding C-glycosylated digitoxin analogues with the various cyclitol-sugars. For example the C-4 ketone can be selectively reduced (e.g., with LiAlH₄, 85-90%) to an allylic alcohol, which in turn can be dihydroxylated (e.g., OsO₄, >80%) to a manno-triol (X, Y, Z=OH). Alternatively, the allylic alcohol products can be reductively rearranged (e.g., NBSH/DIAD/PPh₃) before the dihydroxylation (e.g., OsO₄) to form give a C-2 deoxy-allo-sugar (X═H, Y, Z=OH). By introducing various substitution reactions the hydroxyl groups can be converted into azides and then reduced to amines. One skilled in the art would be able to add X, Y, and Z to the C-2, C-3, and C-4s respectively. X, Y, or Z can be an H, OH, F, Cl, Br, I, NH₂, NHAc, CN, N₃, or OR, where R can be any alkyl, aryl, heteroalkyl, hydroxyalkyl groups with any amino, halo or hydroxyl substitution.

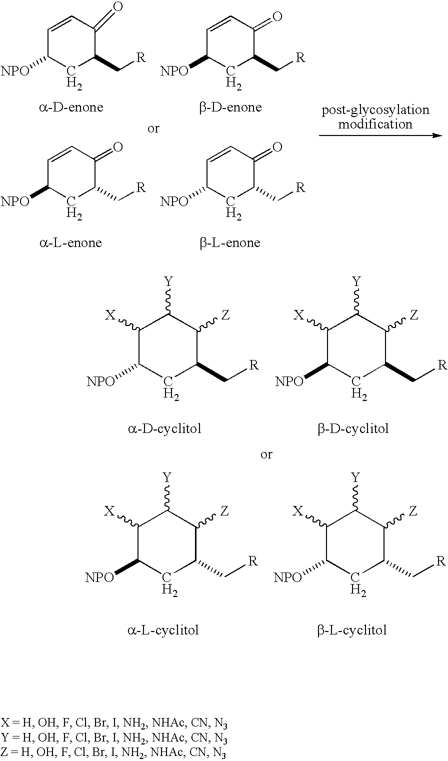

This method is also conducive to the use of digitoxigenin as the NPOH. A palladium catalyzed glycosylation of an alcohol in the NPOH and the corresponding C-1 BocO-enone, which stereospecifically converts the C-1 BocO group to a DigO group. Various post-glycosylation reactions can covert the enone to a corresponding C-glycosylated digitoxin analogue with cyclitol-sugars possessing a myriad of substitution patterns in all the possible stereoisomers. One skilled in the art would be able to add X, Y, and Z to the C-2, C-3, and C-4s respectively. X, Y, or Z can be an H, OH, F, Cl, Br, I, NH₂, NHAc, CN, N₃, or OR, where R can be any alkyl, aryl, heteroalkyl, hydroxyalkyl groups with any amino, halo or hydroxyl substitution.

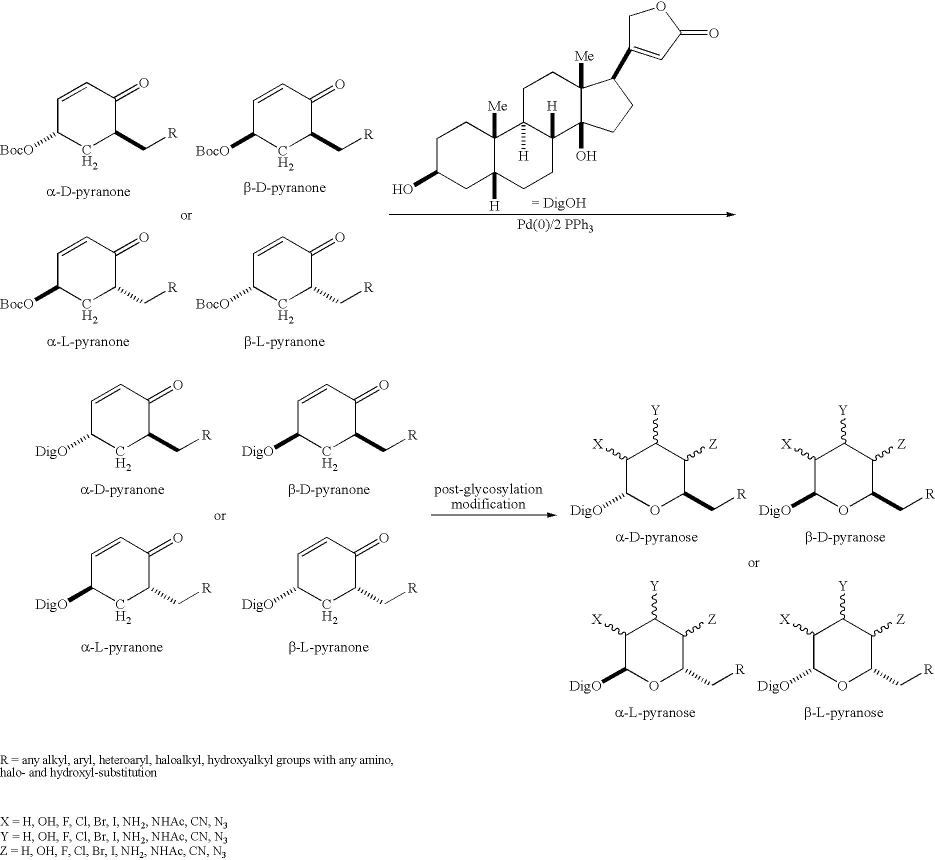

The invention also includes a method for the preparation of C-1 BocO-pyranone as an α/β-mixture from greater than about 4:1 to about 1:1.4 at the C-1 position from the corresponding furan alcohol. The D-pyranones can be prepared from the (R)-furan alcohols and the L-pranones can be prepared from the (S)-furan alcohols by an Achmatowicz oxidation of the furan alcohol followed by Boc-acylation. An Achmatowicz oxidation takes place at about 80-90% yield with a slight excess of NBS in THF/H₂O in about a 4:1 ratio. The Boc-acylation can produce the mixture in greater than about a 4:1 α/β-ratio with (Boc)₂O/DMAP at −78° C.; or in a 1.4:1 α/β-ratio with (Boc)₂O/NaOAc in refluxing benzene at 80° C. The furan alcohols with any substituent can be prepared by the reduction (with >95 % ee) of the corresponding achiral acylfuran, which in turn can be prepared from the corresponding carboxylic acid and furan. When using any substituent R can be any alkyl, aryl, heteroaryl and alkyl group with any amino, halo- and hydroxyl-substitution. Therefore the additional steps of starting with a wanted carboxylic acid and furan and Noyori reduction can give the user the choice of any desired R group.

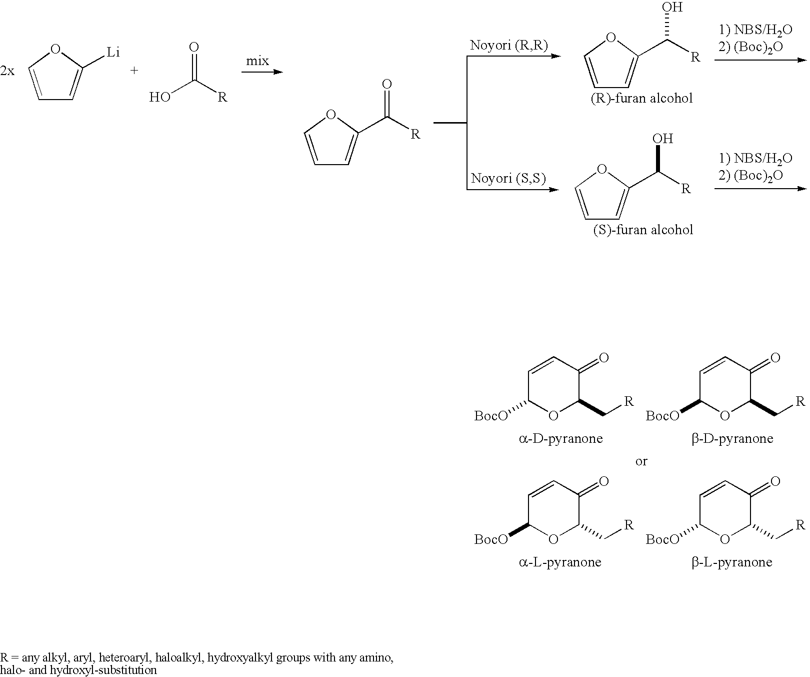

In order to start with a desired C-1 BocO-enone in either of its enantiomeric forms (D/L) and diastereomeric α/β-forms the invention further includes a method of making the desired C-1 BocO-enone from the corresponding quinic acid.

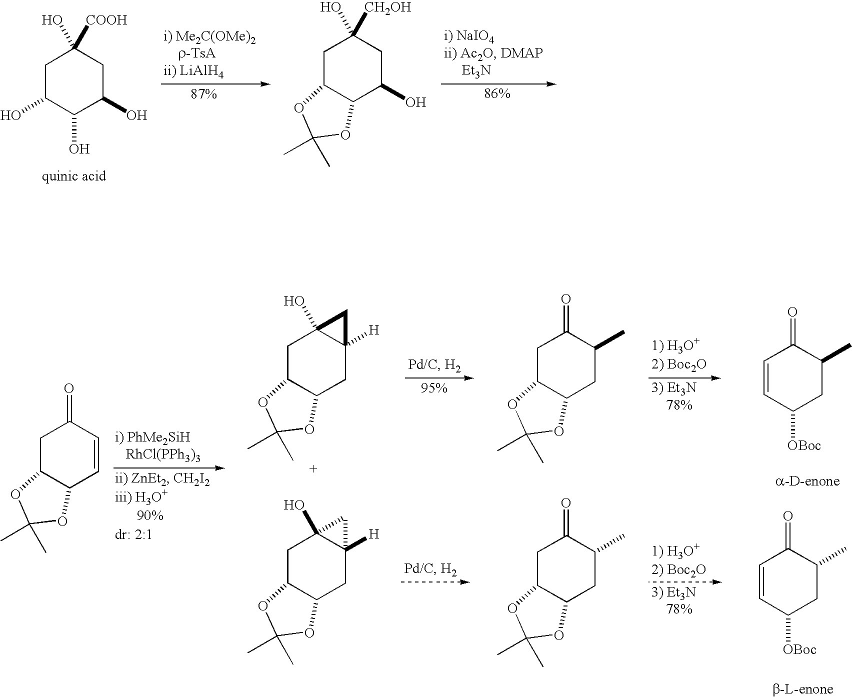

This method is further amenable to a C-6 oxygen substitution as well as synthesis of the L-cyclitol donors from the same enantiomer of the quinic acid.

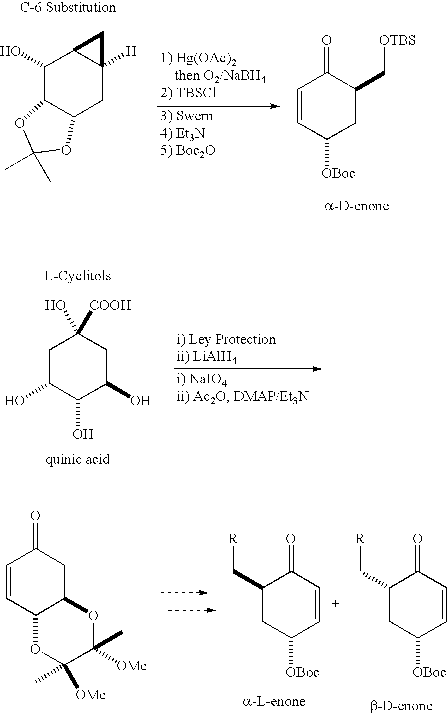

These terms and specifications, including the examples, serve to describe the invention by example and not to limit the invention. It is expected that others will perceive differences, which, while differing from the forgoing, do not depart from the scope of the invention herein described and claimed. In particular, any of the function elements described herein may be replaced by any other known element having an equivalent function.

What is claimed is:

1. A method for the preparation of glycosylated NPOH comprising treating the NPOH with 1-10 equivalents of a BocO-pyranone, catalytic amounts of palladium and catalytic amounts of phosphine ligand wherein an NPO group is stereospecifically placed at the C-1 position of the corresponding pyranone and an R group at the C-6 position can be any alkyl, aryl, heteroalkyl, hydroxyalkyl groups with any amino, halo or hydroxyl substitution.

2. The method for preparation of glycosylated NPOH of claim 1 further comprising post-glycosylation reactions of said pyranone adding C-2 (X), C-3 (Y), and C-4 (Z) chosen from the group of H, OH, F, Cl, Br, I, NH₂, NHAc, CN, and N₃.

3. The method for the glycosylated NPOH of claim 1 wherein said NPOH is digitoxigenin.

4. A compound comprising an unnatural sugar wherein an NPOH is attached to one of 32 possible pyranose stereoisomers at C-1, C-2, C-3, C-4, and C-5 both (R) and (S) wherein the C-2 (X), C-3 (Y), and C-4 (Z) groups are at least one substituent chosen from H, OH, F, Cl, Br, I, NH₂, NHAc, CN, and N₃ and the C-6 (R) is any alkyl, aryl, heteroaryl or alkyl group with any amino, halo or hydroxyl substitution.

5. The compound of claim 4 wherein said natural product is digitoxigenin:

6. A method for the preparation of glycosylated NPOH comprising treating the NPOH with about 1-10 equivalents of a BocO-enone, catalytic amounts of palladium and catalytic amounts of phosphine wherein an NPO group is stereospecifically placed at the C-1 position of the corresponding enone and an R group at the C-6 position can be any alkyl, aryl, heteroalkyl, hydroxyalkyl groups with any amino, halo or hydroxyl substitution.

7. The method for preparation of glycosylated NPOH of claim 6 further comprising post-glycosylation reactions to convert the enone functionality of said pyranone by adding C-2 (X), C-3 (Y), and C-4 (Z) chosen from the group of H, OH, F, Cl, Br, I, NH₂, NHAc, CN, and N₃.

8. The method for the glycosylated NPOH of claim 6 wherein said NPOH is digitoxigenin.

9. A method to prepare a C-1 BocO-pyranone from a furan alcohol comprising using an (R)-furan alcohol to prepare a D-pyranone and an (S)-furan alcohol to prepare an L-pyranone in an α/β-mixture ranging from greater than 4:1 to about 1:1.4 by using an Achmatowicz oxidation of said furan alcohol followed by a Boc-acylation.

10. The method to prepare a C-1 BocO-pyranone from a furan alcohol of claim 9 further comprising the addition of an R group to the C-6 position wherein R is chosen from any alkyl, aryl, heteroalkyl, hydroxyalkyl groups with any amino, halo or hydroxyl substitution wherein said furan is mixed at about twice the amount as a carboxylic acid containing said R group and a Noyori reduction is performed on the resulting achiral acylfuran to yield said (R)-furan alcohol or said (S)-furan alcohol.

11. A method to prepare a C-1 BocO-enone in α/β-form at the C-1 position from the corresponding quinic acid by the following method:

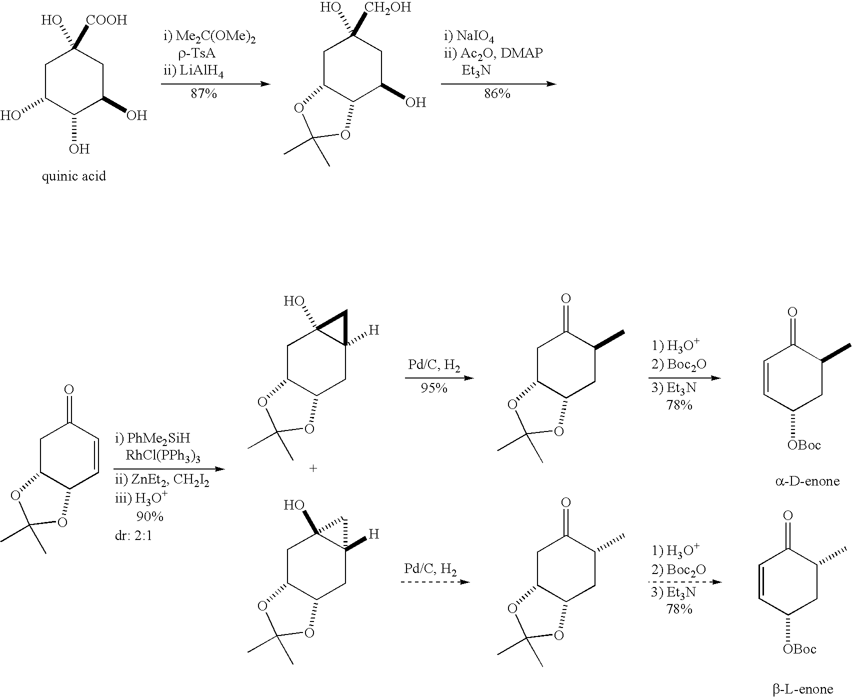

12. The method to prepare a C-1 BocO-enone in α/β-form at the C-1 position from the corresponding quinic acid from claim 11 further comprising a C-6 oxygen substitution.

13. The method to prepare a C-1 BocO-enone in α/β-form at the C-1 position from the corresponding quinic acid from claim 11 further comprising the synthesis of the L-cyclitol donor from the same enantiomer of quinic acid.
